(19) United States
(12) Patent Application Publication
(10) Pub. No.: US 2016/0175443 A1
(43) Pub. Date: Jun. 23, 2016

(54) Treatment options for allergies

(71) Applicant: Michael Lewis Moravitz, Fairfax, VA (US)

(72) Inventor: Michael Lewis Moravitz, Fairfax, VA (US)

(21) Appl. No.: 14/998,792

(22) Filed: Feb. 18, 2016

(51) Int. Cl. A61K 45/00

(52) U.S. Cl. CPC A61K 45/00

(57) ABSTRACT

Treatments for Allergies Previously Thought to be Genetically-based.

Allergies may be based on bacteria and reactions to poisons based on the health of the individual and any weaknesses in their defenses; in other words, the basis would not necessarily be genetic but the condition of the sufferer at that time based on the health of their organs and immune systems. Some allergies do go away over time—perhaps as the sufferer strengthens. Allergies to shellfish, for example, may be based on poisons in the food or bacteria that most would not be susceptible to. Allergies—severe reactions—to insect bites may be related to weaknesses in the body's responses. Research projects should be launched to focus on these possible causes of allergies rather than genetic reasons. This may lead to new treatments for allergies or consideration of the condition and composition of the foods.

One other area to look at is: why was the tomato considered poisonous? Was it just an “Old Wife's Tale” or are their substances in the tomato that caused severe reactions at that time (perhaps more so than now) that caused this idea to be perpetuated in Early Modern Europe. Perhaps tomatoes adapted or became hybrids that lessened the effect of the substance, or people become less susceptible to the substance in the tomato that causes the reaction. This idea may lead to consideration of the causes of allergies to tomatoes, and new treatments.

1) Antihistamines for Allergies;

2) Antibiotics.
